(19) United States
(12) Patent Application Publication
(10) Pub. No.: US 2022/0293230 A1
(43) Pub. Date: Sep. 15, 2022

(54) SYSTEM, APPARATUS, METHOD, AND RECORDING MEDIUM FOR LIMITING FUNCTION OF THERAPEUTIC APPLICATION

(71) Applicant: CureApp, Inc., Tokyo (JP)

(72) Inventor: Masaki Aijima, Chuo-ku, Tokyo (JP)

(73) Assignee: CureApp, Inc., Tokyo (JP)

(21) Appl. No.: 17/635,966

(22) PCT Filed: Aug. 16, 2019
(86) PCT No.: PCT/JP2019/032160

§ 371 Date: Feb. 16, 2022

(51) Int. Cl. G16H 10/60

(52) U.S. Cl. CPC G16H 10/60

(57) ABSTRACT

A system for limiting a function of a therapeutic application for treating a patient who is a user of a user terminal, the system including: a medical record information storage unit configured to record restriction-related information used to determine a function of an application associated with the user based on information generated in connection with first therapeutic application; a to-be-restricted determination unit configured to determine whether a function of a second therapeutic application is to be restricted based on the restriction-related information; and a function restriction unit configured to limit a function of the second therapeutic application that is determined to be restricted.

### TECHNICAL FIELD

The present invention relates to a system, an apparatus, a method, and a program for limiting a function of a therapeutic application.

### BACKGROUND ART

In recent years, a therapeutic application has been developed, and the therapeutic application operates in a user terminal such as a smartphone and is used for improvement of behavior related to health of a user, treatment of a disease, health management, and the like (Patent Document 1). In the system disclosed in Patent Document 1, a system that sequentially provides an individual on a daily basis with a behavior modification message for improving poor behavior related to health, based on data collected from the individual is disclosed.

### CITATION LIST

### Patent Literature

Patent Document 1: JP 2001-92876 A

### SUMMARY OF INVENTION

### Technical Problem

These therapeutic applications are designed for treatment of a disease targeted by each application, and are not considered for interaction with other applications and the like.

### Solution to Problem

The present invention has been made in view of the problems described above, and has characteristics such as the following. That is, a system according to an embodiment of the present invention is a system for limiting a function of a therapeutic application for treating a patient who is a user of a user terminal, and includes a medical record information storage unit configured to record restriction-related information used to determine a function of an application to be restricted, wherein the restriction-related information is associated with the user based on information generated in connection with first therapeutic application, a to-be-restricted determination unit configured to determine whether a function of a second therapeutic application is to be restricted based on the restriction-related information, and a function restriction unit configured to limit a function of the second therapeutic application that is determined to be restricted.

The system includes a server and a user terminal, the server includes at least a portion of the medical record information storage unit, and further includes a restriction-related information transmission unit configured to transmit the restriction-related information to the user terminal at a predetermined timing, and the user terminal may include at least a portion of the to-be-restricted determination unit and the function restriction unit.

The user terminal further includes a restriction-related information request unit configured to transmit a restriction-related information request to the server when executing the second therapeutic application, and the predetermined timing may include a timing at which the server has received the restriction-related information request.

The predetermined timing may include a timing at which the restriction-related information is updated based on information related to the first therapeutic application.

The first and second therapeutic applications are performed at the user terminal, the user terminal may further include at least a portion of the medical record information storage unit configured to record at least a portion of the restriction-related information associated with the first therapeutic application, and a restriction-related information transmission unit configured to transmit the restriction-related information related to the first therapeutic application to the server, and the restriction-related information transmission unit of the server may transmit the restriction-related information request associated with the first therapeutic application to the user terminal at the predetermined timing, and may transmit the restriction-related information to the user terminal based on the restriction-related information associated with the first therapeutic application received from the user terminal.

The system includes a server and a user terminal, the server includes at least a portion of the medical record information storage unit and at least a portion of the to-be-restricted determination unit, and further includes a restriction instruction information transmission unit configured to transmit, to the user terminal, restriction instruction information that instructs restriction of the function of the second therapeutic application determined to be restricted, the user terminal includes the function restriction unit, and the function restriction unit can limit the function of the second therapeutic application determined to be restricted based on the restriction instruction information.

Determining whether the function is to be restricted includes determining whether an identifier indicative of the second therapeutic application is included in the restriction-related information as an identifier of the application that is a target of restriction of use for the user, and limiting the function of the second therapeutic application may include setting the second therapeutic application to not be usable.

The to-be-restricted determination unit may further determine whether the function of the second therapeutic application is to be restricted based on the restriction-related information associated with the second therapeutic application.

Determining whether the function is to be restricted includes determining whether therapeutic information presented to the user using the second therapeutic application is included as restriction target information in the restriction-related information, and limiting the function of the second therapeutic application may execute therapeutic information presentation processing to the user based on the second therapeutic application so as not to include the therapeutic information determined to be included as the to-be-restricted information.

Determining whether the function is to be restricted may include determining whether an execution time for the user to execute an action indicated by therapeutic information defined by the first therapeutic application included in the restriction-related information at least partially overlaps with an execution time for the user to execute an action indicated by therapeutic information defined by the second therapeutic application and comparing, in accordance with a determination that at least a portion of the execution time overlaps, a priority associated with the action defined by the first therapeutic application and a priority associated with the action defined by the second therapeutic application, and changing the therapeutic information defined by the therapeutic application associated with a lower priority such that the execution time does not overlap.

A user terminal according to an embodiment is a user terminal for limiting a function of a therapeutic application for treating a patient who is a user of the user terminal, and includes a medical record information storage unit configured to record restriction-related information associated with the user based on information generated in connection with first therapeutic application, a to-be-restricted determination unit configured to determine whether a function of a second therapeutic application is to be restricted based on the restriction-related information, and a function restriction unit configured to limit a function of the second therapeutic application that is determined to be restricted.

A user terminal according to another embodiment is a user terminal for limiting a function of a therapeutic application for treating a patient who is a user of the user terminal and may include a medical record information storage unit configured to record restriction-related information indicating a function of an application to be restricted, wherein the restriction-related information is associated with the user based on information generated in connection with a first therapeutic application, a restriction-related information transmission unit configured to transmit the restriction-related information to the user terminal at a predetermined timing, a communication unit configured to receive the restriction instruction information determined based on the restriction-related information, and a function restriction unit configured to limit a function of a second therapeutic application based on the restriction instruction information.

A server according to an embodiment is a server for limiting a function of a therapeutic application for treating a patient who is a user of a user terminal, and includes a communication unit configured to receive restriction-related information associated with the user based on information generated in connection with first therapeutic application and transmits restriction instruction information, and a to-be-restricted determination unit configured to determine whether a function of a second therapeutic application is to be restricted based on the received restriction-related information and generates the restriction instruction information.

A method according to an embodiment is a method executed by one or more computers for limiting a function of a therapeutic application for treating a patient who is a user of a user terminal, and includes a step of recording restriction-related information associated with the user based on information generated in connection with first therapeutic application, a step of determining whether a function of a second therapeutic application is to be restricted based on the restriction-related information, and a step of limiting a function of the second therapeutic application that is determined to be restricted.

A program according to one embodiment causes one or more computers to execute the above-described method.

### Advantageous Effects of Invention

By using the present invention, it is possible to limit the function of a therapeutic application that weakens an effect of treatment performed in connection with other therapeutic applications, cannot be executed, and has an adverse effect such as being unusable in relation to a disease and the like targeted by the other therapeutic applications.

### BRIEF DESCRIPTION OF DRAWINGS

FIG. 1 is a configuration diagram of a system according to an embodiment of the present invention.

FIG. 2 is a hardware configuration diagram of a user terminal, a server, and a medical professional terminal according to an embodiment of the present invention.

FIG. 3 is a functional block diagram of the user terminal, the server, and the medical professional terminal according to an embodiment of the present invention.

FIG. 4 is a flowchart according to an embodiment of the present invention.

FIG. 5 is a flowchart according to an embodiment of the present invention.

FIG. 6 is a flowchart according to an embodiment of the present invention.

FIG. 7 is a flowchart according to an embodiment of the present invention.

FIG. 8 is a flowchart according to an embodiment of the present invention.

FIG. 9 is a flowchart according to an embodiment of the present invention.

### DESCRIPTION OF EMBODIMENTS

### First Embodiment

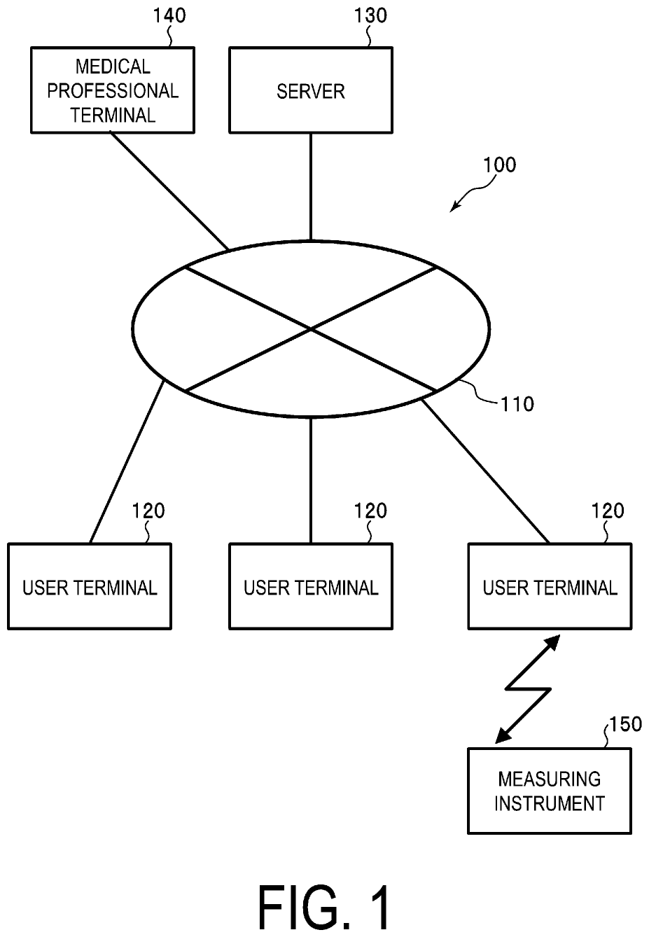

FIG. 1 illustrates an example of a system configuration diagram of the present invention. A system 100 is used for limiting a function of a therapeutic application, and includes a network 110, and a user terminal 120, a server 130 and a medical professional terminal 140 connected to the network 110. In addition, a treatment-related measuring instrument 150 connected to the user terminal 120 may be included. For example, it can include a weight meter for measuring body weight in accordance with an instruction of a carbon monoxide concentration meter or a fatty liver treatment application for performing carbon monoxide concentration measurement in accordance with an instruction of the therapeutic application. The measuring instrument 150 can operate in conjunction with the therapeutic application by, for example, being wirelessly connected to the user terminal 120.

In the present embodiment, although the therapeutic application means an application that improves behavior related to health of the user, or that is used for treatment of a disease or health management, any application may be used as long as the application is used to improve a health condition of the user. The therapeutic application is not limited to an application provided by a medical professional or used under management of a medical professional. Furthermore, each therapeutic application may not have to be created by the same manufacturer, but may be created by two or more manufacturers, or may be downloaded from an application providing server or the like managed by a different person than the manufacturer.

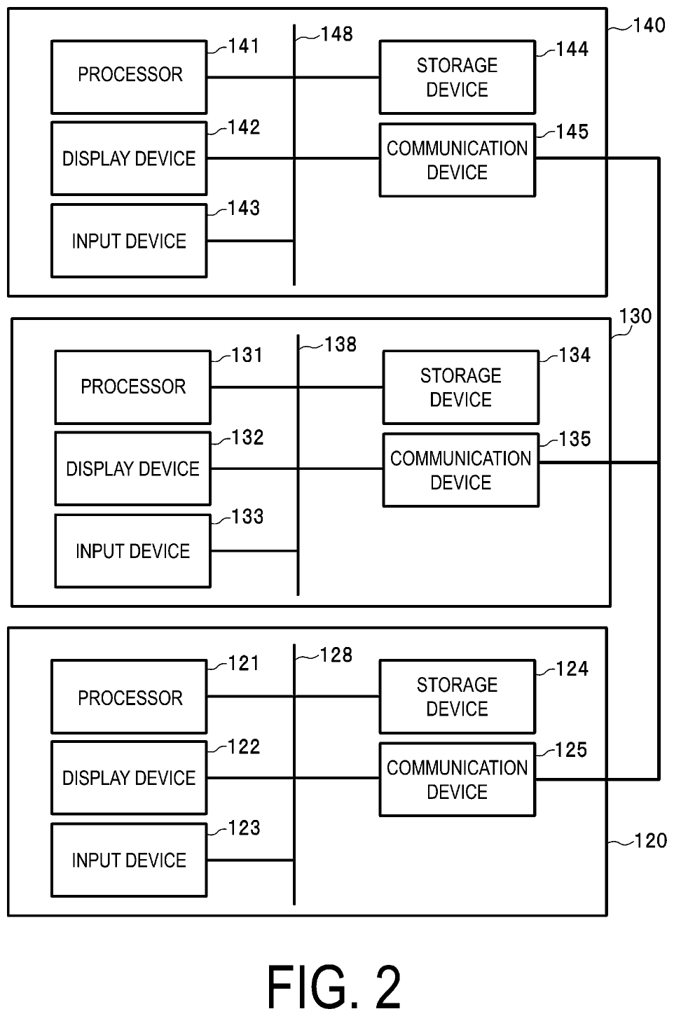

FIG. 2 is a block diagram illustrating a hardware configuration of the user terminal 120, the server 130, and the medical professional terminal 140 according to an embodiment of the present invention. The user terminal 120 includes a processor 121, a display device 122, an input device 123, a storage device 124, and a communication device 125. Each of these component devices is connected by a bus 128. Note that it is assumed that an interface is interposed between the bus 128 and each of the component devices as necessary. In the present embodiment, the user terminal 120 is a smartphone. However, the user terminal 120 can be an information terminal such as a personal computer, a tablet computer, and a touch pad as long as the user terminal 120 includes the configuration describe above.

The server 130 and the medical professional terminal 140 also similarly include processors 131, 141, display devices 132, 142, input devices 133, 143, storage devices 134, 144, and communication devices 135, 145, respectively. Each of these component devices is connected by a bus 138, 148. Note that it is assumed that an interface is interposed between the bus 138, 148 and each of the component devices as necessary. In the present embodiment, the server 130 and the medical professional terminal 140 are realized by a computer.

The processor 121, 131, 141 controls all operations of the user terminal 120, the server 130, and the medical professional terminal 140, and is, for example, a CPU. The processor 121, 131, 141 executes various types of processing by reading and executing a program and data stored in the storage device 124, 134, 144. In an example, the processor 121, 131, 141 includes a plurality of processors.

The display device 122, 132, 142 displays an application screen and the like to a user (patient) of the user terminal 120, a user (administrator) of the server 130, and a user (medical professional) of the medical professional terminal 140, according to control of the processor 121, 131, 141. The display device 122, 132, 142 is preferably a liquid crystal display, but may be a display using organic EL, a plasma display, or the like.

The input device 123, 133, 143 is a user interface that receives an input from the user with respect to the user terminal 120, the server 130, and the medical professional terminal 140, and the input device 123, 133, 143 is, for example, a touch panel, a touch pad, a keyboard, or a mouse. In the present embodiment, the user terminal 120 is a smartphone, and thus, the user terminal 120 includes a touch panel as the input device 123, and the touch panel also functions as the display device 122, and the display device 122 and the input device 123 are integrated. The display device 122 and the input device 123 may be in a separate form in which the display device 122 and the input device 123 are disposed at separate positions. Here, the server 130 and the medical professional terminal 140 are computers, and thus it is assumed that the server 130 and the medical professional terminal 140 each include a keyboard and a mouse as the input device, and a liquid crystal display as the display device.

The storage device 124, 134, 144 is a storage device including a RAM that is a volatile memory and a ROM that is a non-volatile memory, and being provided in a common smartphone or computer. The storage device 124, 134, 144 can also include an external memory. For example, the storage device 124, 144 stores a user application for performing the present embodiment, and the storage device 134 stores a server application. The user application includes a user program of the therapeutic application, and various types of data referred to during execution of the user program. As the therapeutic application for the medical professional terminal 140, an application for operating an electronic medical chart can be used. The user program is started in response to an operation of the user with respect to the user terminal 120, and is executed on an operating system (OS) mounted in advance on the user terminal 120. The application for a server includes a function and various types of data for causing reception of an input from the user and presentation of information to the user to be suitably executed by the user program in each of the user terminals 120 who is a client.

In an example, the storage device 124, 134, 144 includes a main storage device and an auxiliary storage device. The main storage device is a volatile storage medium to and from which information can be written and read at high speed, and is used as a storage area and a work area when the processor 121, 131, 141 processes information. The auxiliary storage device stores various programs, and data used by the processor 121, 131, 141 during execution of each program. The auxiliary storage device is, for example, a hard disk device, but may be any non-volatile storage or nonvolatile memory as long as the non-volatile storage or the nonvolatile memory can store information, and the auxiliary storage device may be removable. The auxiliary storage device stores, for example, an operating system (OS), middleware, an application program, and various types of data that can be referred to in association with execution of these programs.

The communication device 125, 135, 145 transmits and receives data to and from other device via the network 110 (omitted in FIG. 2). For example, the communication device 125, 135, 145 performs mobile communication and wireless communication via a wireless LAN or the like, and is connected to the network 110. The user terminal 120 and the medical professional terminal 140 use the communication devices 125 and 135, respectively, and thus perform communication with the server 130 via a network. The communication device 125, 135, 145 may perform wired communication using an Ethernet (trade name) cable or the like.

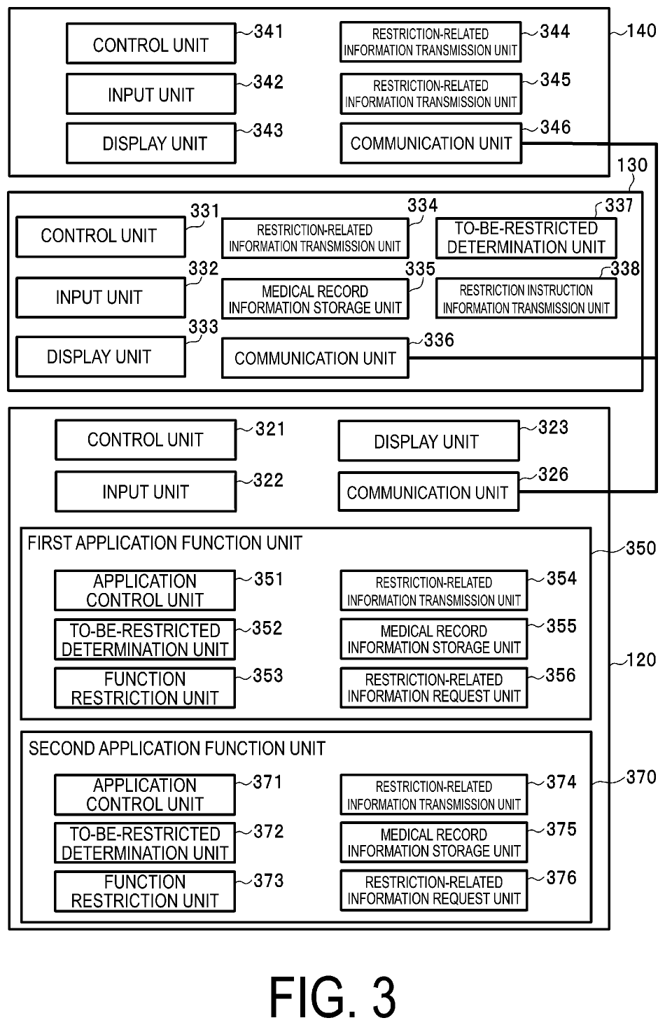

FIG. 3 shows an example of a functional block diagram of the user terminal 120, the server 130, and the medical professional terminal 140 according to an embodiment of the present invention. The user terminal 120 includes a control unit 321, an input unit 322, a display unit 323, a communication unit 326, a first application function unit 350, and a second application function unit 370, the server 130 includes a control unit 331, an input unit 332, a display unit 333, a restriction-related information transmission unit 334, a medical record information storage unit 335, a communication unit 336, and a to-be-restricted determination unit 337, and the medical professional terminal 140 includes a control unit 341, an input unit 342, a display unit 343, a restriction-related information transmission unit 344, a medical record information storage unit 345, and a communication unit 346.

In the present embodiment, these functions are realized by causing the processor 121, 131, 141 to execute a program. For example, the program executed is a program stored in the storage device 124, 134, 144. The first application function unit 350 and the second application function unit 370 are implemented by being executed by the first and second therapeutic applications. In this way, various types of functions are realized by reading a program, and thus, a portion or all of one part (function) may be provided in other part. These functions may be realized by hardware by constituting an electronic circuit or the like for realizing a part or all of each function.

The control unit 321 of the user terminal 120 performs control processing of information processing executed in the present embodiment. The input unit 322 receives an input from the user of the user terminal 120. The display unit 323 displays an application screen for controlling a function of an application, and displays an application screen corresponding to a function of an application and a user operation. In the present embodiment, a touch panel including the input unit 322 and the display unit 323 is used, and an operation receiving unit is realized by a touch detection function. The communication unit 326 performs communication with other user terminal 120, the server 130, the medical professional terminal 140, and the like by wired and wireless communication.

The first and second application function units 350, 370 include application control units 351, 371, to-be-restricted determination units 352, 372, function restriction units 353, 373, restriction-related information transmission units 354, 374, medical record information storage units 355, 375, and restriction-related information request units 356, 376, respectively.

The application control unit 351, 371 performs control processing of information processing executed by each therapeutic application in the present embodiment. The to-be-restricted determination unit 352, 372 determines whether the function of the therapeutic application is to be restricted based on restriction-related information included in medical record information. The function restriction unit 353, 373 limits the function of the therapeutic application determined to be restricted. The limited functions may be the entire therapeutic application, or may be some functions of the therapeutic application. For example, it is assumed that some of the treatment processing executed by the therapeutic application cannot be executed, and the treatment processing can be changed to other executable treatment processing.

The restriction-related information transmission unit 354, 374 has a function to transmit the restriction-related information. The restriction-related information is information used to determine the function of the therapeutic application to be limited, and may include information indicating an application that is not to be used for the user, information identifying an application to be installed and an installed application, information indicating treatment processing that is not to be performed on the user, and information indicating a time that the execution time should not overlap with other treatment processing. In the present embodiment, although the restriction-related information transmission unit 354, 374 transmits the restriction-related information in response to a restriction-related information request, the restriction-related information may be transmitted at a predetermined timing. For example, when a predetermined time is reached, when application registration information is registered, or when the restriction-related information is updated, the restriction-related information may be transmitted.

The medical record information storage unit 355, 375 records the medical record information associated with the user based on information generated in connection with the therapeutic application. The medical record information can include, for example, the gender, age, weight, medication history, past medical history, treatment history, and the like of the user who is a patient. The medical record information can further include the restriction-related information used to determine a limited function of the therapeutic application. The restriction-related information request unit 356, 376 transmits the restriction-related information request that is a signal for requesting the restriction-related information.

The control unit 331 of the server 130 performs processing for an application executed in the user terminal 120 and the medical professional terminal 140. In one example, when the application is executed in the user terminal 120, the control unit 331 transmits and/or receives data periodically, or as necessary, and causes the user terminal 120 to realize the function of the application. The input unit 332 receives an input from the user of the server 130. The display unit 333 displays a management screen for a server administrator as necessary on the display device 132.

The restriction-related information transmission unit 334 has a function to transmit the restriction-related information. The medical record information storage unit 335 records the medical record information associated with the user based on information generated in connection with the therapeutic application. The medical record information can include restriction-related information. The communication unit 336 performs communication with the user terminal 120, the medical professional terminal 140, and the like by wired and wireless communication.

The control unit 341 of the medical professional terminal 140 performs processing for an application executed in the medical professional terminal 140, for example, an electronic medical chart application. The input unit 342 receives an input from the user of the medical professional terminal 140. The display unit 343 displays the application screen for controlling the function of the application, and displays the application screen corresponding to the function of the application and the user operation. The communication unit 346 performs communication with the user terminal 120, the server 130, other medical professional terminal 140, and the like by wired and wireless communication.

The restriction-related information transmission unit 344 has a function to transmit the restriction-related information. The medical record information storage unit 345 records the medical record information associated with the user based on information generated in connection with the therapeutic application. The medical record information can include the restriction-related information. The communication unit 346 performs communication with other user terminal 120, the server 130, the medical professional terminal 140, and the like by wired and wireless communication.

The medical record information storage unit 335, 345, 355, 375 of the user terminal 120, the server 130, and the medical professional terminal 140 can also store the medical record information including the restriction-related information input based on each therapeutic application, may be in a mode in which all of the medical record information are recorded and managed in the medical record information storage unit 345 of the server 130, and in the user terminal 120 and the medical professional terminal 140, only some of the medical record information are recorded and managed or no medical record information is recorded and managed, or may be in a mode in which in the server 130, the restriction-related information transmitted from the one application function unit can be transmitted to the other therapeutic application function unit and only some of the restriction-related information are recorded and managed or no restriction-related information is recorded and managed.

The to-be-restricted determination unit of the present system can be provided in a plurality of devices. In the present embodiment, although the to-be-restricted determination unit is included in the first application function unit 350 and the second application function unit 370 of the user terminal 120, the to-be-restricted determination unit is included in the server 130 in an embodiment described below. The to-be-restricted determination unit may be provided in both the user terminal 120 and the server 130. Similarly, the medical record information storage unit can be distributed and disposed in a plurality of devices including the user terminal 120, the server 130, and the medical professional terminal 140. In this sense, it may be expressed that each device includes at least a portion of the to-be-restricted determination unit, and it may be expressed that at least a portion of the medical record information storage unit is included. Other components can also be distributed and arranged in a plurality of devices. Even if it is not described as “at least a portion”, it can mean that a component is a portion when distributed and disposed in a plurality of devices, and it does not mean that other devices do not always include the configuration.

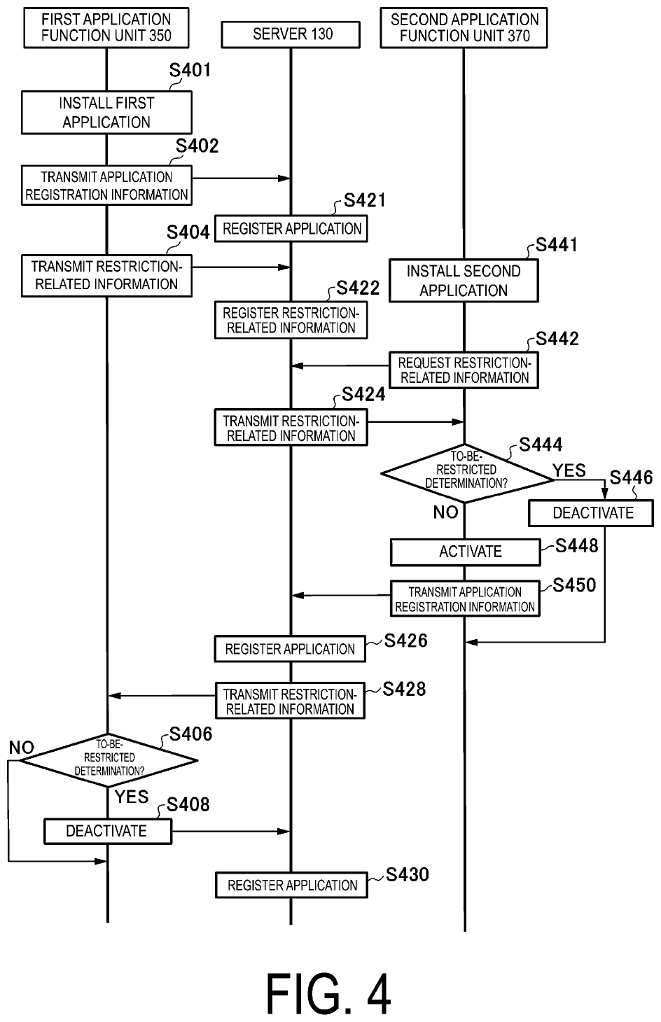

Next, a flow of processing in the present embodiment will be described with reference to FIG. 4. In this case, a case where a user using a therapeutic application for depression further starts to use an application for smoking cessation treatment will be described as an example; however, it does not matter what kind of disease as long as the therapeutic applications are those that weaken an effect of treatment performed in connection with other therapeutic applications, cannot be executed, and have an adverse effect such as being unusable in relation to a disease and the like targeted by the other therapeutic applications.

The disease does not need to be a disease in a medical sense as long as the disease is in a physically or mentally unpreferable state in addition to a medical disease such as renal dysfunction, hypertension, or mental disease. The treatment may be any treatment that is intended to improve a physically or mentally unpreferable state, and may be preventive medical care. The patient refers to a person who attempts to improve physical condition using the present invention, and does not have to be a patient who is treating a disease under the guidance of a medical professional.

In FIG. 4, the first application function unit 350 for executing a function of a first therapeutic application (first application) that is an application for depression treatment and the second application function unit 370 for executing a function of a second therapeutic application (second application) that is an application for smoking cessation treatment are described separately for explanatory purposes; however, in the present embodiment, it is assumed that the application function units are installed and implemented in one user terminal 120. The application function units may be separately installed in two user terminals 120 and implemented at each user terminal.

First, the user who is a patient suffering from depression is prescribed the application for depression treatment as the first application from a physician, and installs the application on the user terminal 120 of the user (S401). The control unit 351 of the first application function unit 350 implemented by the installation transmits the application registration information to the server 130 (S402). The application registration information includes, for example, a user ID for identifying the user, an application ID of the first application, and the restriction-related information. The application ID may be a portion of the restriction-related information.

The application ID of the first application may be stored in the application control unit 351 of the first application function unit during installation of the first application, or may be stored in the medical record information storage unit 355 as a portion of the restriction-related information of the first application. The application ID can be, for example, an identifier for the application assigned by an application providing site prepared to make applications of a plurality of manufacturers downloadable. The application ID may be an identifier assigned for each application in advance to make the system available.

The restriction-related information can include the application ID of another therapeutic application that opposes treatment associated with the first therapeutic application. The application registration information may be transmitted including the medical record information other than the restriction-related information. During installation, the restriction-related information may be stored in the medical record information storage unit 355 of the first application function unit. In addition, the medical record information other than the restriction-related information can be stored in the medical record information storage unit 355.

There is a case where it is determined that a predetermined application for smoking cessation treatment should not be used for a patient with depression according to determination of a medical professional, and in this case, the application ID of the predetermined application for smoking cessation treatment for which it is determined that the use is to be restricted for the patient with depression is included as the restriction-related information. The application for smoking cessation treatment should not always be restricted for the patient with depression. All applications for smoking cessation treatment or other therapeutic applications may be used, and the use of a predetermined therapeutic application may be restricted. The restriction-related information is assumed to be set in advance to the first application. The restriction-related information may be set in formulating the therapeutic application from a physician.

The server 130 performs application registration of the user based on the received application registration information (S421). In the application registration, the user ID and the application ID of the first application can be associated and stored in a user-to-application table included in the medical record information stored in the medical record information storage unit 335, and the user ID and the restriction-related information can be associated and stored in a user-to-restriction-related information table. In the present embodiment, as illustrated in Tables 1 and 2 below, the application ID of a depression application is registered as the first application, and the application ID of a predetermined application for smoking cessation treatment whose use is to be restricted as the restriction-related information is registered.

TABLE 1
User-to-application table

| User ID | Application ID | Status |
|---|---|---|
| 0809 | 0521 | Active |
| 0028 | 0928 | Active |
| . . . | . . . | |

TABLE 2
User-to-restriction-related information table

| | | Restriction-related information |
|---|---|---|
| User ID | Application ID | (Restriction target application ID) |
| 0809 | 0521 | 0888 |
| 0028 | 0928 | 0571 |
| . . . | | . . . |

In Tables 1 and 2, user ID=0028 is the user ID of the user using the present embodiment, and Application ID=0928 is the application ID of the application for depression treatment, and Application ID=0571 is the application ID of the predetermined application for smoking cessation treatment for which it is determined that the use is to be restricted. In Table 1, User IDs=0809 and 0028 indicate that the applications of Application IDs=0521 and 0928 are used, respectively. If the status is active, it indicates that the application is still installed and used, and if the status is inactive, it means that the application is not currently used, such as if the application is uninstalled. For an application that has stopped being used, the application ID of the application may be deleted from the user-to-application table. Table 2 shows that, for the application for depression treatment of Application ID=0928 used by User ID=0028, the application for smoking cessation treatment of Application ID=0571 is to be restricted. When the application ID is made inactive in the user-to-application table, the restriction-related information related to the application ID is deleted in a user-to-restriction-related information table.

Based on an input operation to the user terminal 120 as a response to a medical interview by the first application, the user transmits the new restriction-related information together with the user ID and the application ID to the server 130 (S404), and the server 130 that has received the new restriction-related information stores the user ID and the restriction-related information in association with each other in the medical record information storage unit 335. When the restriction-related information is transmitted, the restriction-related information may be stored in the medical record information storage unit 355 of the first application function unit. The medical record information obtained as a response to the medical interview may be stored in the medical record information storage unit 355.

In addition, when the user installs the second application on the user terminal 120 (S441), the restriction-related information request unit 376 of the second application transmits the restriction-related information request together with the user ID (S442). Here, the installation is not limited to a case where the therapeutic application is completely installed, and also includes a case where a function capable of executing to-be-restricted determination processing is installed before the application executes therapeutic processing.

When the server 130 receives the restriction-related information request, the server 130 reads the restriction-related information associated with the user ID from the user-to-restriction-related information table of the medical record information storage unit 335, and transmits the restriction-related information to the second application function unit 370 (S424). In the present embodiment, the application ID of a predetermined application for smoking cessation treatment whose use is to be restricted associated with the user ID is read and transmitted to the second application function unit 370.

The to-be-restricted determination unit 372 of the second application function unit 370 that has received the application ID determines whether the second application is an application to be restricted based on the received restriction-related information (S444). For example, the application ID included in the restriction-related information is compared with the application ID of the second application, and when the application IDs match, the second application is determined to be the application to be restricted. In the present embodiment, the restriction-related information includes the application ID of a predetermined application for smoking cessation treatment, and when the application ID is compared with the application ID of the second application, the application IDs match, so that the second application is determined to be the application to be restricted. The application ID of the second application may be stored in the application control unit 371 of the second application function unit during installation of the second application, or may be stored in the medical record information storage unit 375 as a portion of the restriction-related information of the second application.

In accordance with a determination that the second application is the application to be restricted, the second application is deactivated (S446). Deactivation means setting an application to an unavailable state, and includes not only disabling an installed application but also interrupting installation, uninstalling, and the like.

As the deactivation, while the application may be temporarily set to the unavailable state, the application may be activated by permission input by a medical professional or the like. For example, if the application is deactivated, a message that “You need physician's permission to use this application. Please consult a physician” is displayed on the display unit 323 of the user terminal 120, in accordance with a determination that the application is usable after diagnosis by the physician, the physician can operate the user terminal 120 and input a passcode or the like to perform permission input, and the user terminal 120 can receive the input and activate the application. After the application is activated, the processing in and after S450 can be executed. In addition, for example when a permission request signal for requesting permission is transmitted to the medical professional terminal 140 and the user terminal receives a permission response signal transmitted from the medical professional terminal based on input by the medical professional, the application is activated, and the application can be activated based on information indicating permission of the medical professional or the like. By adopting such an aspect, for example, even a user suffering from depression can use a predetermined application for smoking cessation treatment with the permission by the medical professional, assuming that the application is not always limited. The restriction-related information may include use restriction level information, and whether the application is always prohibited to be used or can be used based on the permission of the physician may be determined based on a use restriction level. For example, if the use restriction level=1, after deactivation, activation may be allowed based on the permission input by the physician, and if the use restriction level=2, deactivation may be made, so that it is possible not to accept the permission input by the physician.

In accordance with a determination that the second application is not to be restricted, the second application is activated (S448). The restriction-related information may be stored in the medical record information storage unit 375 of the second application function unit. The activation means to make the application available, and includes, after only limited functions are installed, completion of installation of remaining functions, and the like. When installation has already been completed and the application is in the available state, additional processing may not be executed.

In addition, similarly to the installation of the first application, the application registration information for the second application is transmitted (S450). The application registration information includes the user ID, the application ID of the second application, and the restriction-related information. The server 130 that has received the application registration information performs application registration (S426). That is, in the user-to-application table and the user-to-restriction-related information table, the application ID of the second application and the restriction-related information are stored in association with the user ID. When the application registration information is transmitted, the restriction-related information may be stored in the medical record information storage unit 375 of the second application function unit. In addition, other medical information related to the second application may be stored.

At a timing when new application information is stored in association with the user ID in the medical record information storage unit 345 of the server 130, the restriction-related information transmission unit 344 of the server 130 transmits the new restriction-related information to the first application function unit 350 that is the application function unit of another application already registered in association with the user ID. The restriction-related information can be transmitted at a timing when the restriction-related information for the user is updated according to new application registration information.

The to-be-restricted determination unit 352 of the first application function unit determines whether the first application is to be restricted by the same procedure as the second application function unit 370 based on the received restriction-related information (S406). When the application ID of the first application matches the application ID included in the restriction-related information, the first application is an application whose use is restricted, and is deactivated (S408), and when the application ID of the first application is not included, the first application is kept active.

When the first application has been deactivated, information indicating that the first application has been deactivated is transmitted to the server 130 (S408), and the status of the first application associated with the user ID in the medical record information in the server 130 is changed to inactive. Alternatively, information related to the application ID of the deactivated application may be deleted.

In the present embodiment, although the individual application ID is used as the restriction-related information, an application type ID that identifies an application type can also be used. For example, when there are a plurality of applications for smoking cessation treatment, an application type ID is included in the application ID, and a type ID indicating the type of the application for smoking cessation treatment, which is the application type ID, can be used as the restriction-related information. Then, the to-be-restricted determination processing can be executed by comparing the type ID included in the application ID with the type ID that is the restriction-related information. The same applies to other embodiments.

In the present embodiment, when the first application is installed, the to-be-restricted determination processing is not executed. However, prior to the application registration information transmission (S402), processing similar to S442, 424, and 444 to 448 may be executed to execute the to-be-restricted determination processing.

In the present embodiment, when the restriction-related information request is received from the application function unit, and when a new application and the restriction-related information are stored in the medical record information storage unit of the server 130 in association with the user ID, the restriction-related information is transmitted to the application control unit. The restriction-related information can be transmitted at other predetermined timings, and for example, the restriction-related information may be transmitted at a predetermined time. The restriction-related information may be transmitted in response to the application registration information instead of the response to the restriction-related information request.

By using the present embodiment, the therapeutic application that weakens the effect of treatment performed in connection with other therapeutic applications and adversely affects a disease or the like targeted by another therapeutic application and whose use is to be restricted can be made unavailable. By preventing execution of the therapeutic application whose predetermined use is to be prohibited, it is possible to prevent the user from falling into a serious disease state or to prevent adverse effects on other treatments in advance.

In the present embodiment, the application determined to be restricted is deactivated; however, as a modification, an application having a low priority may be deactivated based on a score indicating the priority of each application. For example, the score is included in each application registration information, and in accordance with a determination that the second application is included in the restriction-related information of the first application in the to-be-restricted determination processing, the scores of the first and second applications are compared.

If the score of the second application is lower, the second application is deactivated. When the score of the first application is lower, a deactivation instruction for deactivating the first application is transmitted to the second application via the server 130 while the second application is activated. The second application that has received the deactivation instruction can execute deactivation processing.

This makes it possible to give priority to use of a therapeutic application with higher priority and contribute to health improvement of the user who is a patient.

As another modification, in accordance with a determination that the first application or the second application is the application to be restricted, information indicating that the first application or the second application is the application to be restricted may be presented to the user based on the determination result, or the determination result may be transmitted to the medical professional terminal 140 to be presented to the medical professional, and the medical professional may select which of the first and second applications is to be deactivated. The selected application can be activated based on selected input to the user terminal or the medical professional terminal 140 by the user or medical professional.

As a result, use of a suitable application for the user is selected by the user or the medical professional, and it is possible to contribute to health improvement of the user who is a patient.

As a further modification, information indicating only whether there is the therapeutic application already in use by the user is used as the restriction-related information, and when there is the therapeutic application in use, use of another therapeutic application may be restricted. In this case, when the first application is registered, the first application function unit 350 may transmit the application ID of the first application as the application registration information, and the server 130 may register that the first application is being used as the application registration (S421). However, it is sufficient to register that the user is using any therapeutic application. It is not necessary to transmit the first application ID from the first application function unit 350, and information indicating which application is a target of restriction of use is also not necessary. The server 130 does not need to store which application the user is using. When processing of installing the second application is started, the restriction-related information transmission unit 334 of the server 130 transmits information indicating that the user is already using the therapeutic application as the restriction-related information (S424). In accordance with a determination that the restriction-related information indicates that there is an application in use, the second application is determined to be restricted (S444), and deactivation is performed (S446). Accordingly, when one therapeutic application is being used, the use of another therapeutic application can be prohibited.

### Second Embodiment

A second embodiment is different from the first embodiment in that the application ID of an application being used by a user and the application ID of another application being used for restricting an application on a restricted side are used as the restriction-related information instead of using the application ID of an application to be restricted. Hereinafter, a difference from the first embodiment will mainly be described.

In the present embodiment, as in the first embodiment, a first application is installed, and the application is registered in a server 130. The restriction-related information in this case is the application ID of the first application installed and being used by the user. In this case, since new restriction-related information is not registered, S404 and S422 may be omitted.

When a second application is installed, a restriction-related information request is made (S422), and the application ID of the first application is transmitted as the restriction-related information from the server 130 (S424). The second application includes, as the restriction-related information, the application ID of the second application and the application ID in use for restricting the second application. In this case, during installation of a predetermined application for smoking cessation treatment, it is assumed that the restriction-related information of the second application includes the application ID of the first application that is an application for depression treatment as the application ID that is being used for restricting the application.

In the to-be-restricted determination processing (S444), a to-be-restricted determination unit 372 of a second application function unit 370 can determine whether or not the second application is to be restricted based on the received restriction-related information and the restriction-related information of the second application that is a target of the to-be-restricted determination processing. For example, the in-use application ID included in the received restriction-related information is compared with the application ID of the in-use application for restricting the second application, and the second application is determined to be restricted when the application IDs match. In this case, since the first application that is a predetermined application for depression treatment in which a predetermined application for smoking cessation treatment is to be restricted is being used, the second application that is the application for smoking cessation treatment is determined to be restricted, and the second application is deactivated (S446).

In accordance with a determination that the second application is not to be restricted, the second application is activated (S448), the application ID of the second application is transmitted to the server 130 as a portion of the application registration information (S450), the application is registered (S426), and the application ID of the second application is transmitted as the restriction-related information to a first application function unit 350 (S428). The case of transmitting the restriction-related information includes a case where only a portion of the restriction-related information is transmitted.

Similarly to the second application, the first application can determine whether the first application is to be restricted by comparing the application ID of the second application that is the application in use with the application ID in use for restricting the first application in the restriction-related information of the first application.

In the present embodiment, the restriction-related information transmitted from the application function unit to the server is only the application ID of the own application; however, the restriction-related information may be transmitted together with the application ID in use for the own application to be restricted. In a mode as described below in which the to-be-restricted determination unit in the server determines whether to be restricted, it is possible to perform to-be-restricted determination based on these pieces of information.

### Third Embodiment

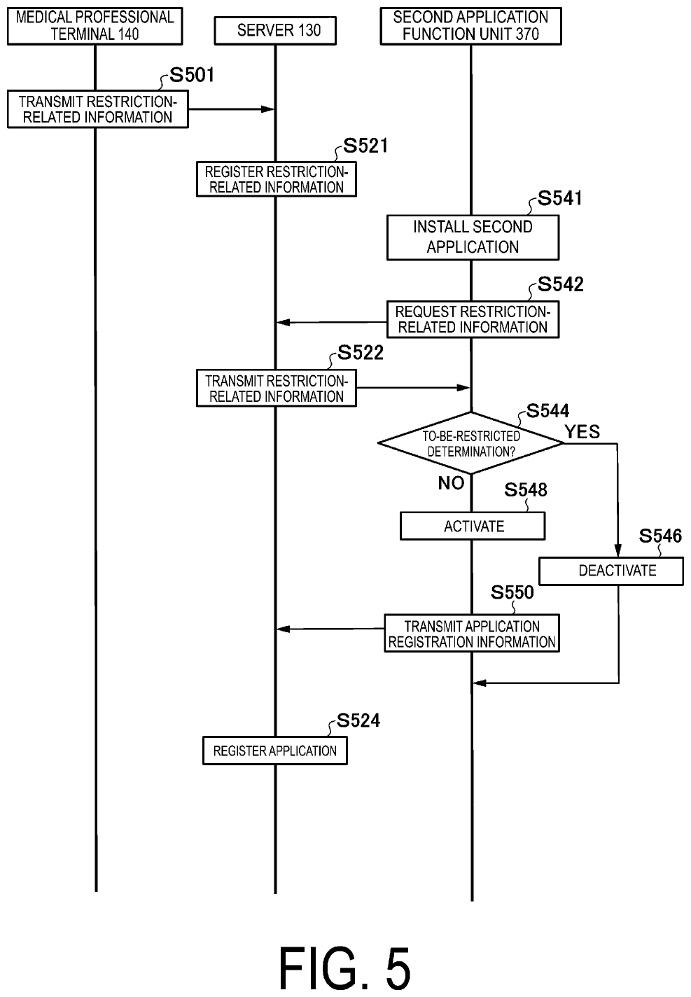

As illustrated in FIG. 5, a third embodiment is different from the first embodiment in that a medical professional terminal 140 transmits restriction-related information to a server 130 instead of the first application function unit 350. Hereinafter, a difference from the first embodiment will mainly be described.

In the present embodiment, it is assumed that an electronic medical record application is installed as a first application on the medical professional terminal 140. Then, the restriction-related information is generated based on information input to the electronic medical record application by the medical professional through a medical interview with a patient who is a user of a user terminal 120, and the restriction-related information is transmitted to the server 130 together with a user ID and an application ID (S501). For example, the restriction-related information is generated based on medical record information such as the gender, age, weight, medication history, past medical history, treatment history, and the like of the user who is the patient, which is input to the medical professional terminal 140 and stored in a medical record information storage unit 345 that stores the restriction-related information.

Similarly to the first embodiment, the server 130 associates the restriction-related information with the user ID based on the received restriction-related information, and stores the restriction-related information as a user-to-restriction-related information table similar to Table 2 in a medical record information storage unit 335. In this case, it is assumed that the application ID of the electronic medical record application is registered with respect to the user ID in the user-to-application table. The electronic medical record application is not used in the user terminal 120, and thus may not be registered in the user-to-application table.

Then, in the user terminal 120, when the second application is installed, it is determined whether the application is to be restricted based on the restriction-related information received in response to a restriction-related information request according to a procedure similar to that of the first embodiment. In accordance with a determination that the application is to be restricted, the application is deactivated, and in accordance with a determination that the application is not to be restricted, application registration is executed (S524, 5541 to S550).

For example, when a physician inputs that the patient suffers from depression together with the user ID through the electronic medical record application and stores the information in the medical record information storage unit 345 at the time of a medical interview with the physician in outpatient clinic for depression, the electronic medical record application generates an application ID of a predetermined application for smoking cessation treatment, which is an application whose use is to be restricted and stored in association with a disease ID that identifies depression, as the restriction-related information and transmits the generated application ID to the server 130. Then, when the user attempts to install the application for smoking cessation treatment as the second application, the restriction-related information including the application ID for smoking cessation treatment is received, the application for smoking cessation treatment is determined to be an application to be restricted by determination whether to be restricted based on the restriction-related information, and the application is disabled.

As the restriction-related information, the disease ID may be transmitted from the medical professional terminal 140 to the server 130, a table of the unavailable application ID for the disease ID may be stored in the server 130, the application ID of an application whose use for the disease ID is to be restricted may be determined based on the table, and the determined application ID may be stored in a user-to-restriction target table.

By using the present embodiment, even when the restriction-related information is transmitted via a therapeutic application installed in the medical professional terminal 140 different from the user terminal 120, similarly to the first embodiment, it is possible to prevent an application that adversely affects the user from being intruded, and to prevent the user from falling into a serious disease state in advance.

As in the second embodiment, it is also possible to use the restriction-related information indicating the disease ID for restricting the application on the restricted side. For example, when the physician inputs that the patient suffers from depression together with the user ID through the electronic medical record application and stores the information in the medical record information storage unit 345 of the medical professional terminal 140, a depression ID is transmitted as the restriction-related information to the server 130. The depression ID is included as the restriction-related information of the second application, which is the application for smoking cessation treatment, and the to-be-restricted determination unit compares the depression ID, which is the restriction-related information related to the first application, with the depression ID, which is the restriction-related information related to the second application, and determines that the second application is the application to be restricted since these depression IDs match.

When there is a disease that is already being treated, installation of the therapeutic application may be prohibited. For example, when the physician inputs that the patient suffers from depression together with the user ID through the electronic medical record application and stores the information in the medical record information storage unit 345 of the medical professional terminal 140, the depression ID is transmitted as the restriction-related information to the server 130, and the server 130 records information indicating that there is a disease being treated in a medical record information storage unit 355. The server 130 transmits, to a second application function unit 370, the information indicating that there is a disease being treated as the restriction-related information (S522), and the second application function unit 370 can deactivate the application (S546) when there is the disease being treated.

### Fourth Embodiment

A fourth embodiment is different from the first embodiment in that the to-be-restricted determination unit of the first and second application function units is not used, and a to-be-restricted determination unit 337 of the server 130 executes to-be-restricted determination processing instead of the application function unit. Hereinafter, a difference from the first embodiment will mainly be described.

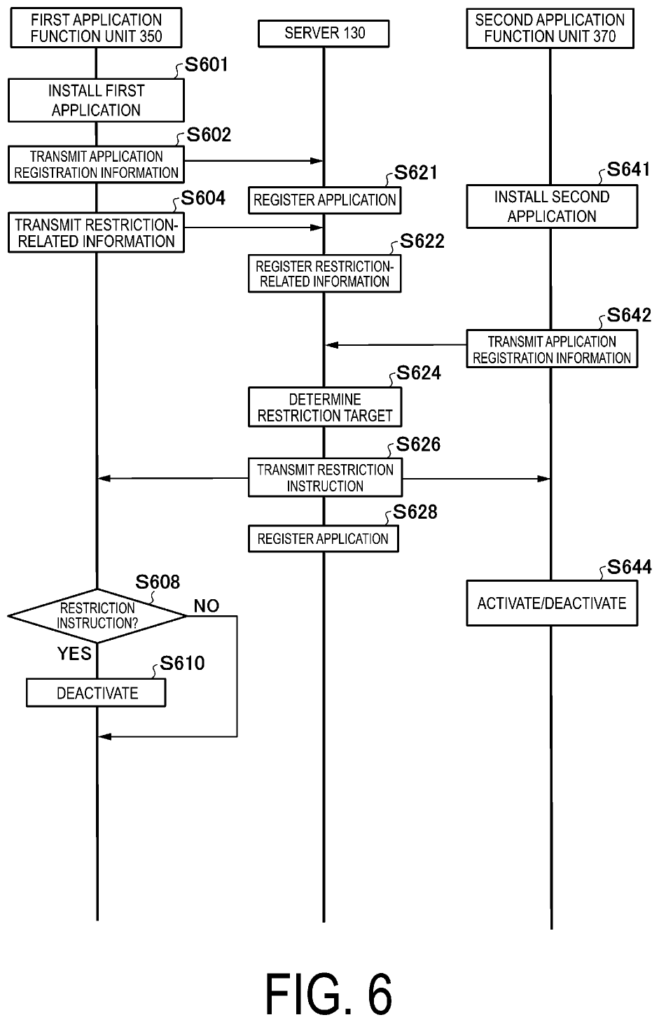

As illustrated in FIG. 6, when the first application is installed in the user terminal 120 and the restriction-related information is transmitted, application registration and restriction-related information registration are performed in the server 130 (S601 to 5604, S621 to S622), and when the second application is installed in the user terminal 120, application registration information is transmitted to the server 130 without making a restriction-related information request (S642).

The to-be-restricted determination unit 337 of the server 130 executes the to-be-restricted determination processing based on medical record information stored in a medical record information storage unit 335 and the application registration information transmitted from the second application function unit 370 (S624). More specifically, the restriction-related information in a user-to-restriction-related information table is read based on a user ID included in the application registration information transmitted from the second application function unit 370, and it is determined whether the restriction-related information includes information indicating the application ID of the second application included in the application registration information transmitted from the second application function unit 370.

In accordance with a determination that the information indicating the application ID of the second application is included in the restriction-related information of the medical record information, it is determined that the second application is an application to be restricted, and in accordance with] a determination that the information is not included, it is determined that the second application is not an application to be restricted.

In addition, in the present embodiment, in accordance with a determination that the second application is not to be restricted, it is determined whether the first application is to be restricted. The application registration information transmitted from the second application function unit 370 includes the restriction-related information, and the to-be-restricted determination unit 337 reads information indicating the application ID stored in association with the user ID in a user-to-application table stored in the server 130. It is determined whether the read application ID is included in the restriction-related information from the second application function unit 370.

A score indicating a priority may be given to the first and second applications, and may be included in the application registration information, score comparison processing of the first and second applications may be performed prior to the to-be-restricted determination processing, and it may be determined whether the application is to be restricted in order from the application having a lower score. Alternatively, information indicating that the first and second applications are the applications to be restricted may be presented on a display unit 323 of the user terminal 120, and which of the first and second applications is to be deactivated via an input unit 322. Determination result information may be transmitted to the medical professional terminal 140, displayed on a display unit 343 of the medical professional terminal, and selected by the medical professional.

After the to-be-restricted determination processing is executed, a restriction instruction information transmission unit 338 of the server transmits restriction instruction information to the application function unit based on the determination result. Information indicating an activation or deactivation instruction is transmitted to the second application function unit 370, and the second application function unit 370 activates or deactivates the second application based on the information (S644).

On the other hand, the deactivation instruction can be transmitted to a first application function unit 350 only when a determination result is that the first application function unit 350 is deactivated. Then, the first application function unit 350 that has received the restriction instruction information indicating the deactivation instruction executes deactivation of the first application (S608, S610).

After the server 130 has transmitted the restriction instruction information, the server 130 executes application registration processing (S628). When the second application is activated, the user ID and the application ID of the second application are registered in association with each other. Even when it is determined to deactivate the second application, the user ID and the application ID of the second application may be registered in association with each other, and the status may be registered as inactive. When the first application is deactivated, the application ID of the first application stored in association with the user ID in the medical record information may be deleted, or the status may be made inactive.

By using the present embodiment, since the to-be-restricted determination processing is executed in the server 130, a processing load on the user terminal 120 can be reduced.

### Fifth Embodiment

The fifth embodiment is different from the first embodiment in which the application itself is deactivated in that a portion of the functions of the application is restricted. Here, it is assumed that therapeutic restriction processing is performed as function restriction processing. Hereinafter, a difference from the first embodiment will mainly be described. In the present embodiment, it is assumed that the first and second applications are already installed in the user terminal 120 and activated. It is assumed that the first and second applications are an application for hypertension treatment and an application for fatty liver treatment, respectively.

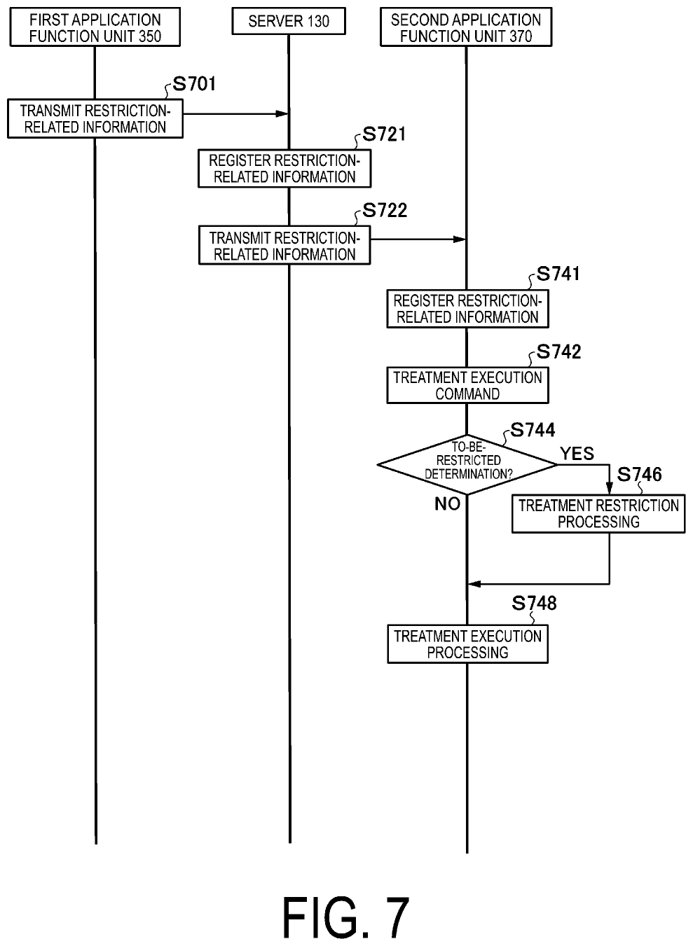

As illustrated in FIG. 7, in a medical interview function of the application for hypertension treatment, which is the first application, information indicating that the user is taking an anti-coagulant agent X, which is one of anti-coagulant agents for hypertension treatment, is input via the user terminal 120, and the application for hypertension treatment stores, as medical history information, the information indicating the anti-coagulant agent X in a medical record information storage unit 355. The restriction-related information is generated based on the medical history information and transmitted to the server 130 (S701).

For example, as illustrated in Table 3, a first application function unit 350 stores information, indicating medicines, ingredients, nutrients, and the like that are not preferably used in combination with the anti-coagulant agent X, as restriction target information in association with the medicine ID indicating the anti-coagulant agent X, and transmits to-be-restricted information, indicating the unpreferable medicines, ingredients, nutrients, and the like, as the restriction-related information to the server 130 based on input of information indicating that the anti-coagulant agent X is being taken.

TABLE 3
Medicine-to-restriction-related information table

| Medicine ID | Restriction target ID |
|---|---|
| 0001 | 0582, 0923, 1200 |
| 0002 | 2305 |
| . . . | . . . |

In Table 3, a to-be-restricted ID is associated with the medicine ID. The restriction target ID is a to-be-restricted ID that identifies an ingredient or the like to be restricted. It is preferable that the to-be-restricted ID is shared in the entire system. However, when a portion using a different identification form is included, for example, the to-be-restricted ID can be mutually converted by a conversion table or the like and used.

It is assumed that Medicine ID=0001 is ID of the anti-coagulant agent X, and Restriction target IDs=0582, 0923, and 1200 as the restriction-related information are IDs corresponding to natto, green juice, and a high-vitamin K ingredient, respectively. In this case, it is assumed that the effect of the anti-coagulant agent X is reduced when the high-vitamin K ingredient is ingested, and the high-vitamin K ingredient is not good for patients taking the anti-coagulant agent X. Therefore, information indicating these unpreferable ingredients is registered in Table 3 for the anti-coagulant agent X.

In the present embodiment, based on the medical history information indicating that the patient is taking the anti-coagulant agent X included in the medical record information, IDs indicating natto, green juice, and the high-vitamin K ingredient are read from Table 3 based on the ID of the anti-coagulant agent X, and are transmitted to the server 130 as the restriction-related information. In the server 130, the restriction-related information received in association with the user ID is stored in the medical record information storage unit 335 (S721), and the restriction-related information is transmitted to the second application function unit 370 (S722). In the server 130, for example, as the user-to-restriction-related information table described in Table 2, the medical record information storage unit 335 can store the application ID of the first application and the to-be-restricted ID as the restriction-related information in association with the user ID.

When the second application function unit 370 that implements the application for fatty liver treatment as the second application receives the restriction-related information, the second application function unit 370 stores the restriction-related information in a medical record information storage unit 375 (S741). Thereafter, therapeutic processing is executed at a predetermined timing (S742). The predetermined timing can be, for example, a timing at which the user feels appetite, starts the second application, touches a nurse call button displayed on the display unit 323 of the user terminal 120, and obtains advice from the second application.

The second application determines whether appetite impulse is to be suppressed based on the time at that time, and in accordance with a determination that it is not necessary, it is determined to execute therapeutic processing by displaying an instruction message that “You have a proper appetite, so you may have a proper meal. It is good to eat high-protein and low-calorie ingredients, for example, natto” on the display unit 323.

The second application function unit 370 determines whether the therapeutic processing to be executed corresponds to to be restricted (S744). In the present embodiment, it is determined whether the instruction message determining execution includes information included in the restriction-related information. In this case, since Restriction target ID=0582 indicating natto is included in the restriction-related information and “natto” is included in the instruction message, the instruction message is determined to be restricted. In this determination, for example, in the second application function unit 370, the to-be-restricted ID included in the instruction message is stored in association with an instruction message ID that identifies each instruction message, and the determination can be made by comparing with the to-be-restricted ID stored in the medical record information storage unit 375.

In accordance with a determination that the therapeutic processing to be executed is to be restricted, the therapeutic restriction processing is executed (S746). In this case, the instruction message to be presented to the user is changed to another instruction message that “You can take the meal, but when you eat, eat slowly and thoroughly.” that does not include the restriction-related information, and the instruction message is displayed on the display unit 323, so that alternative therapeutic processing is executed and therapeutic information presentation processing is performed (S748). In accordance with a determination that the therapeutic processing is not to be restricted, the therapeutic processing is executed by displaying the instruction message determined by the second application function unit 370 on the display unit 323 (S748).

By the therapeutic restriction processing, the therapeutic processing may not be executed at the timing. The therapeutic processing to be executed may not be determined before the to-be-restricted determination processing, and the therapeutic processing to be executed may be selected and executed from the therapeutic processing that is not to be restricted by excluding the therapeutic processing to be restricted in advance based on the restriction-related information.

A person skilled in the art naturally understands that the present embodiment can be executed by a similar procedure even in a case where the restriction-related information is transmitted to the server 130 via the electronic medical record application in the medical professional terminal 140 instead of the first application function unit 350 realized in the user terminal 120. For example, when it is input that the user has a nut allergy via the electronic medical record application, as the restriction-related information, the to-be-restricted ID indicating the nut can be registered in the server 130 in association with the user ID, and can be transmitted to the second application function unit 370. Then, guidance information including nuts is restricted from being displayed, and the therapeutic restriction processing can be executed so that other guidance information is displayed.

By using the present embodiment, when two or more therapeutic applications are used in combination, it is possible to perform treatment intended by each application while limiting the function of the therapeutic application that weakens the effect of treatment performed in connection with other therapeutic applications and adversely affects such as being contraindicated for a disease or the like targeted by another therapeutic application.

In the present embodiment, the server 130 stores the restriction-related information, received from the first application function unit 350, in the medical record information storage unit 335 of the server. However, the restriction-related information may be temporarily stored, may be transmitted to the second application function unit 370, and may not be stored and held in the server 130. The present invention can also be realized even by the server 130 having a small storage region.

As the restriction-related information, information indicating the medical record information such as medical history information and information indicating to be restricted function associated with the medical history information or the like for restricting an application on the restricted side can be used, instead of using information indicating an unpreferable medicine, ingredient, nutrient, or the like. For example, it is possible to determine the function to be restricted in the second application by using the medicine ID being taken as the restriction-related information of the first application and using a table as illustrated in Table 3 indicating an unpreferable medicine, ingredient, nutrient, or the like to be restricted related to the second application as the restriction-related information of the second application.

### Sixth Embodiment

The sixth embodiment is different from the fifth embodiment in that the restriction-related information is transmitted in response to the restriction-related information request. Hereinafter, a difference from the fifth embodiment will mainly be described.

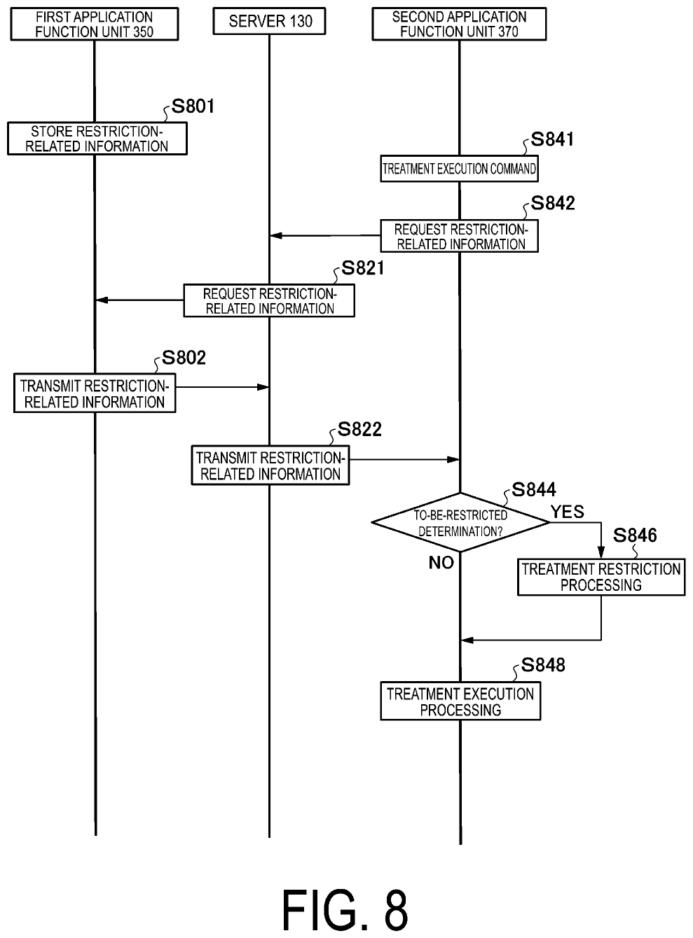

As illustrated in FIG. 8, in the medical interview function of the application for hypertension treatment, which is the first application, the medical history information indicating that the user is taking the anti-coagulant agent X for hypertension treatment is input via the user terminal 120, the application for hypertension treatment generates the restriction-related information based on the information indicating the anti-coagulant agent X, and the restriction-related information is stored in the medical record information storage unit 355 of the first application function unit 350 (S801).

When the second application function unit 370 that implements the application for fatty liver treatment as the second application executes the therapeutic processing based on a treatment execution command at a predetermined timing (S841), the second application function unit 370 transmits the restriction-related information request to the server 130 (S842). The server 130 that received the restriction-related information request further transmits the restriction-related information request to the first application function unit 350 (S821). When the first application function unit 350 receives the restriction-related information request, the first application function unit 350 transmits the restriction-related information to the server 130 (S802), and the server 130 further transmits the restriction-related information to the second application function unit (S822).

The second application function unit performs the to-be-restricted determination, the treatment restriction processing, and the treatment execution processing according to a procedure similar to that of the fifth embodiment based on the received restriction-related information (S844 to S848).

By using the present embodiment, the server does not need to store and manage the restriction-related information for all the user IDs, and server load can be reduced.

### Seventh Embodiment

The seventh embodiment is different from the fifth embodiment in that execution time overlapping detection processing is performed as the to-be-restricted determination processing and overlapping resolution processing is executed as function restriction processing. Hereinafter, a difference from the fifth embodiment will mainly be described. In the present embodiment, similarly to the third embodiment, the server 130 uses the to-be-restricted determination unit 337. It is assumed that first and second applications are already installed in a user terminal 120 and activated. It is assumed that the first and second applications are an application for smoking cessation treatment and an application for fatty liver treatment, respectively.

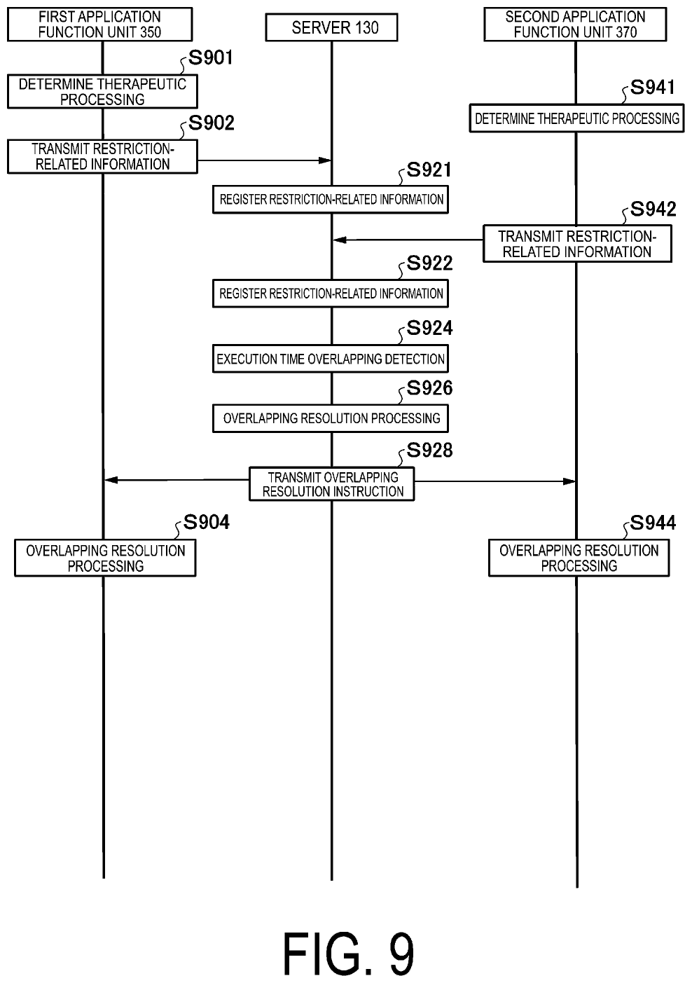

As illustrated in FIG. 9, the treatment processing to be executed by the first application is determined (S901), the restriction-related information based on the treatment processing is transmitted to the server 130 (S902), and the server 130 registers the restriction-related information as the restriction-related information (S921). For example, through the medical interview function of the application for smoking cessation treatment which is the first application, as one smoking cessation treatment, a task of writing a diary before 7:30 which is a morning commuting time and from 7:00 to 7:30 is determined (S901), and a task ID indicating task contents, a task assignment time, and a score are transmitted to the server 130 together with the user ID as therapeutic information included in the restriction-related information. When the server 130 receives the therapeutic information, the server 130 registers the restriction-related information in association with the user ID in the medical record information storage unit 335 (S921).

Similarly, the second application also determines the therapeutic processing to be executed (S941), the restriction-related information based on the therapeutic processing is transmitted to the server 130 (S942), and the server 130 registers the restriction-related information as the therapeutic information included in the restriction-related information (S922). In the present embodiment, as one of the fatty liver treatments, a task of advancing an attendance time in the morning by 30 minutes and walking in one station section for 30 minutes, that is, walking from 7:00 to 7:30 is assigned and transmitted as the restriction-related information.

In this case, it is assumed that the medical record information storage unit 335 of the server 130 stores a user-to-task table illustrated in Table 4 as the restriction-related information.

TABLE 4
User-to-task table

| User ID | Application ID | Task ID | Allocation time | Score |
|---|---|---|---|---|
| 0028 | 0571 | 0235 | 0700:0730 | 30 |
| 0028 | 2878 | 0215 | 0700:0730 | 25 |
| . . . | . . . | . . . | . . . | . . . |

The score is a value indicating the priority of the task, and a higher score means a higher priority. For example, a higher score is given for a more medically important task. A value can be determined in advance for each score by a medical professional or the like. Adjustment and determination may be appropriately made according to a state of the user based on the medical record information.

The to-be-restricted determination unit 337 of the server 130 determines whether the execution time of one task overlaps with the execution time of another task based on the restriction-related information at a predetermined timing (S924). The predetermined timing can be, for example, a timing at which a new task is registered in the user-to-task table, or may be a predetermined time such as 12:00 every day.

In the present embodiment, when the user-to-task table includes one or more tasks for one user ID and a new task is registered for the user ID, the execution time overlapping detection processing is executed. The execution time overlapping detection processing is executed by transmitting the restriction-related information by the first application, registering the first task in the task table, and registering the new task in the task table to which the restriction-related information by the second application has been transmitted.

The to-be-restricted determination unit 337 of the server 130 compares the assignment times of the tasks assigned to the same user ID with reference to the user-to-task table, and determines whether there is an overlap. Here, the overlap can include not only a case where the times completely overlap but also a case where some times overlap.

When the overlap is detected, overlapping resolution processing is executed as the function restriction processing (S926). Here, the overlapping resolution processing includes, for example, processing of canceling one task while maintaining the other task, changing the other task to another time, or changing to another task executed at another time. At this time, which task is to be maintained can be determined based on the score. For example, a task with a higher score can be maintained, and a task with a lower score can be cancelled. The overlap can be eliminated by shifting or shortening the times of both tasks.

In this case, it is detected that a task “write a diary” in which Task ID=0235 and a task “walk” in which Task ID=0215 overlap at Assignment time=0700: 0730 (S924). Since the score of the task “write a diary” is 30 and the score of the task “walk” is 25, the task “write a diary” is maintained as it is, and the task “walk” is changed to another task having no overlapping execution time (S926). In the server 130, information on the task “walk” is deleted from the user-to-task table, and the changed other task is newly registered.

The overlapping resolution processing is executed, and an overlapping resolution instruction is transmitted to the application to which the task ID to be changed is assigned (S928). In this case, since the task “walk” is determined as the task to be changed, the overlapping resolution instruction is transmitted to the second application function unit 370 to which the task “walk” is assigned.

The application function unit that has received the overlapping resolution instruction executes the overlapping resolution processing (S904, S944). In this case, for example, the task “walk” may be simply canceled, or the execution time of the task “walk” may be changed to 18:00 to 18:30 at the time of coming home.

By using the present embodiment, when two or more therapeutic applications are used in combination, it is possible to perform treatment intended by each application while limiting the function of the therapeutic application that weakens the effect of treatment performed in connection with other therapeutic applications and adversely affects such as being contraindicated for a disease or the like targeted by another therapeutic application.

As a modification, as the overlapping resolution processing, information indicating that the tasks overlap is transmitted to the user terminal 120 or the medical professional terminal 140 and is presented to the user or the medical professional, and any task can be selected.

As a result, use of a suitable application for the user is selected by the user or the medical professional, and it is possible to contribute to health improvement of the user who is a patient.

In the processing or operations described above, the processing or operations can be modified freely as long as there is no occurrence of contradiction in the processing or operations such as utilization at a certain step of data that still cannot be utilized at that step. Additionally, each example described above is exemplified for describing the present invention, and the present invention is not limited to these examples. The present invention can be performed in various forms without departing from the spirit of the present invention.

### REFERENCE SIGNS LIST

100 System
110 Network
120 User terminal
121 Processor
122 Display device
123 Input device
124 Storage device
125 Communication device
128 Bus
130 Server
131 Processor
132 Display device
133 Input device
134 Storage device
135 Communication device
138 Bus
140 Medical professional terminal
141 Processor
142 Display device
143 Input device
144 Storage device
145 Communication device
148 Bus
150 Measuring instrument
321 Control unit
322 Input unit
323 Display unit
326 Communication unit
331 Control unit
332 Input unit
333 Display unit
334 Restriction-related information transmission unit
335 Medical record information storage unit
336 Communication unit
337 To-be-restricted determination unit
338 Restriction instruction information transmission unit
341 Control unit
342 Input unit
343 Display unit
344 Restriction-related information transmission unit
345 Medical record information storage unit
346 Communication unit
350 First application function unit
351 First application control unit
352 To-be-restricted determination unit
353 Function restriction unit
354 Restriction-related information transmission unit
355 Medical record information storage unit
356 Restriction-related information request unit
370 Second application function unit
371 Application control unit
372 To-be-restricted determination unit
373 Function restriction unit
374 Restriction-related information transmission unit
375 Medical record information storage unit
376 Restriction-related information request unit

1. A system for limiting a function of a therapeutic application for treating a patient who is a user of a user terminal, the system comprising:
a medical record information storage unit configured to record restriction-related information used to determine a function of an application to be restricted, wherein the restriction-related information is associated with the user based on information generated in connection with first therapeutic application;
a to-be-restricted determination unit configured to determine whether a function of a second therapeutic application is to be restricted based on the restriction-related information; and
a function restriction unit configured to limit a function of the second therapeutic application that is determined to be restricted.

2. The system according to claim 1, further comprising:
a server; and
a user terminal, wherein
the server includes at least a portion of the medical record information storage unit, and further includes a restriction-related information transmission unit configured to transmit the restriction-related information to the user terminal at a predetermined timing, and
the user terminal includes at least a portion of the to-be-restricted determination unit and the function restriction unit.

3. The system according to claim 2, wherein
the user terminal further includes a restriction-related information request unit configured to transmit a restriction-related information request to the server when executing the second therapeutic application, and
the predetermined timing includes a timing at which the server has received the restriction-related information request.

4. The system according to claim 2, wherein
the predetermined timing includes a timing at which the restriction-related information is updated based on information related to the first therapeutic application.

5. The system according to claim 2, wherein
the first and second therapeutic applications are performed at the user terminal,
the user terminal further includes at least a portion of the medical record information storage unit configured to record at least a portion of the restriction-related information associated with the first therapeutic application,
a restriction-related information transmission unit configured to transmit the restriction-related information related to the first therapeutic application to the server, and
the restriction-related information transmission unit of the server transmits the restriction-related information request associated with the first therapeutic application to the user terminal at the predetermined timing, and transmits the restriction-related information to the user terminal based on the restriction-related information associated with the first therapeutic application received from the user terminal.

6. The system according to claim 1, further comprising:
a server; and
a user terminal, wherein
the server includes at least a portion of the medical record information storage unit and at least a portion of the to-be-restricted determination unit, and further includes a restriction instruction information transmission unit configured to transmit, to the user terminal, restriction instruction information that instructs restriction of the function of the second therapeutic application determined to be restricted,
the user terminal includes the function restriction unit, and
the function restriction unit limits the function of the second therapeutic application determined to be restricted based on the restriction instruction information.

7. The system according to claim 1, wherein
determining whether the function is to be restricted includes determining whether an identifier indicative of the second therapeutic application is included in the restriction-related information as an identifier of the application that is a target of restriction of use for the user, and
limiting the function of the second therapeutic application includes setting the second therapeutic application to not be usable.

8. The system according to claim 1, wherein
the to-be-restricted determination unit further determines whether the function of the second therapeutic application is to be restricted based on the restriction-related information associated with the second therapeutic application.

9. The system according to claim 1, wherein
determining whether the function is to be restricted includes determining whether therapeutic information presented to the user using the second therapeutic application is included as restriction target information in the restriction-related information, and
limiting the function of the second therapeutic application executes therapeutic information presentation processing to the user based on the second therapeutic application so as not to include the therapeutic information determined to be included as the to-be-restricted information.

10. The system according to claim 1, wherein
determining whether the function is to be restricted includes:
determining whether an execution time for the user to execute an action indicated by therapeutic information defined by the first therapeutic application included in the restriction-related information at least partially overlaps with an execution time for the user to execute an action indicated by therapeutic information defined by the second therapeutic application,
comparing, in accordance with a determination that at least a portion of the execution time overlaps, a priority associated with the action defined by the first therapeutic application and a priority associated with the action defined by the second therapeutic application, and
changing the therapeutic information defined by the therapeutic application associated with a lower priority such that the execution time does not overlap.

11. A user terminal for limiting a function of a therapeutic application for treating a patient who is a user of the user terminal, the user terminal comprising:
a medical record information storage unit configured to record restriction-related information associated with the user based on information generated in connection with first therapeutic application;
a to-be-restricted determination unit configured to determine whether a function of a second therapeutic application is to be restricted based on the restriction-related information; and
a function restriction unit configured to limit a function of the second therapeutic application that is determined to be restricted.

12. A user terminal for limiting a function of a therapeutic application for treating a patient who is a user of the user terminal, the user terminal comprising:
a medical record information storage unit configured to record restriction-related information indicating a function of an application to be restricted, wherein the restriction-related information is associated with the user based on information generated in connection with a first therapeutic application;
a restriction-related information transmission unit configured to transmit the restriction-related information to the user terminal at a predetermined timing;
a communication unit configured to receive the restriction instruction information determined based on the restriction-related information; and
a function restriction unit configured to limit a function of a second therapeutic application based on the restriction instruction information.

13. A server for limiting a function of a therapeutic application for treating a patient who is a user of a user terminal, the server comprising:
a communication unit configured to receive restriction-related information associated with the user based on information generated in connection with first therapeutic application and transmits restriction instruction information; and
a to-be-restricted determination unit configured to determine whether a function of a second therapeutic application is to be restricted based on the received restriction-related information and generates the restriction instruction information.

14. A method performed by one or more computers for limiting a function of a therapeutic application for treating a patient who is a user of a user terminal, the method comprising:
recording restriction-related information associated with the user based on information generated in connection with first therapeutic application;
determining whether a function of a second therapeutic application is to be restricted based on the restriction-related information; and
limiting a function of the second therapeutic application that is determined to be restricted.

15. A computer-readable medium storing a program executed by one or more computers to perform the method according to claim 14.
